(19) United States
(12) Patent Application Publication
(10) Pub. No.: US 2022/0096357 A1
(43) Pub. Date: Mar. 31, 2022

(54) HYDRATING NO-RINSE COMPOSITION FOR MAKEUP REMOVAL

(71) Applicant: L'OREAL, Paris (FR)

(72) Inventor: Catherine CHIOU, Saddle Brook, NJ (US)

(73) Assignee: L'OREAL, Paris (FR)

(21) Appl. No.: 17/038,943

(22) Filed: Sep. 30, 2020

(51) Int. Cl. A61K 8/891; A61K 8/31; A61K 8/34; A61Q 1/14

(52) U.S. Cl. CPC A61K 8/891; A61K 8/31; A61K 2800/87; A61Q 1/14; A61K 8/345

(57) ABSTRACT

A makeup removal composition includes at least one of each of a silicone-based emulsifier, a branched, liquid alkane, an emollient ester, ether, or carbonate, a hydrating agent, and water. In some embodiments, the composition may further include one or more additives including preservatives, actives, and fragrances.

### FIELD

This invention relates to a hydrating no-rinse makeup removal composition.

### BACKGROUND

Removal of stubborn makeup has always been a challenge and a consumer pain point, particularly for mascara and waterproof mascara, long-wear foundation and longer-lasting lip products. Typical makeup removal products are ineffective in removing stubborn makeup, thus requiring repeated tugging and rubbing on the skin and/or the delicate eye areas, causing irritation. Some oil-based makeup removing products may provide ease of makeup removal but leave behind an unpleasant and greasy skin feel. Less greasy alternatives smudge and leave makeup residue. Many of the existing and compositions also require rinsing after use. Accordingly, there is a need for compositions that will provide easy and efficacious makeup removal in addition to a clean and pleasant skin feel after the use and do not require subsequent washing or rinsing.

### SUMMARY

To address the deficiencies in the art with makeup removal products (MURs), the inventors provide a hydrating no-rinse (leave on) makeup removal composition that provides easy and efficacious makeup removal and a soft and smooth skin feel after the use.

The disclosure provides, in various embodiments, a makeup removal composition, comprising:
i. at least one silicone-based emulsifier;
ii. at least one branched, liquid alkane with carbon chain length of C12 to C20;
iii. at least one emollient ester, ether, or carbonate with carbon chain length of C12 to C24;
iv. at least one hydrating agent; and
v. water.

In various embodiments, the composition may include more than one of each of the components, In some embodiments, the composition may further include one or more additives including preservatives, actives, and fragrances.

In accordance with some embodiments:
i. the at least one silicone-based emulsifier is present from about 0.5% to about 20%, by weight of the composition;
ii. the at least one branched, liquid alkane is present from about 1% to about 15% by weight of the composition;
iii. the at least one emollient ester, ether, or carbonate is present from about 0.5% to about 10% by weight of the composition;
iv. the at least one hydrating agent is present from about 10% to about 20% by weight of the composition; and
v. water is present from about 60% to about 85% by weight of the composition.

In accordance with some embodiments:
i. the at least one silicone-based emulsifier is chosen from dimethicone/PEG-10/15 crosspolymer, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and lauryl PEG-9 polydimethylsiloxyethyl dimethicone, and combinations thereof;
ii. the at least one branched, liquid alkane has a carbon chain length of C12 to C20;
iii. the at least one emollient ester, ether, or carbonate has a carbon chain length of C12 to C24 and comprises a dialkylated ether or dialkylated carbonate; and
iv. the at least one hydrating agent comprises a polyol.

In accordance with some embodiments:
i. the at least one silicone-based emulsifier comprises dimethicone/PEG-10/15 crosspolymer, dimethicone/polyglycerin-3 crosspolymer, cetyl PEG/PPG-10/1 dimethicone, or PEG-18/PPG-18 dimethicone, or dimethicone/PEG-10/15 crosspolymer, dimethicone/polyglycerin-3 crosspolymer, cetyl PEG/PPG-10/1 dimethicone, PEG-18/PPG-18 dimethicone, or a combination thereof and PEG-10 dimethicone, PEG/PPG-18/18 methicone, lauryl PEG/PPG-18/18 methicone, PEG-9 polydimethylsiloxyethyl dimethicone, lauryl PEG-9 polydimethylsiloxyethyl dimethicone, or a combination thereof;
ii. the at least one branched, liquid alkane has a carbon chain length of C12 to C20 and comprises isohexadecane, isododecane, C15-19 alkane, or a combination thereof;
iii. the at least one emollient ester, ether, or carbonate has a carbon chain length of C12 to C24 and comprises isopropyl myristate, isopropyl palmitate, ethylhexyl palmitate, dicaprylyl ether, dicaprylyl carbonate, or a combination thereof;
iv. the at least one hydrating agent comprises glycerin.

In accordance with some embodiments:
i. the at least one silicone-based emulsifier comprises dimethicone/PEG-10/15 crosspolymer;
ii. the at least one branched, liquid alkane with carbon chain length of C12 to C20 comprises isododecane or C15-19 alkane;
iii. the at least one emollient ester, ether, or carbonate with carbon chain length of C12 to C24 comprises isopropyl palm itate, ethylhexyl palmitate, dicaprylyl ether, dicaprylyl carbonate, a combination of isopropyl palm itate and dicaprylyl ether, or a combination of isopropyl palm itate and dicaprylyl carbonate; and
iv. the at least one hydrating agent comprises glycerin.

In accordance with some embodiments:
i. the at least one silicone-based emulsifier comprises dimethicone/PEG-10/15 crosspolymer and PEG-10 dimethicone;
ii. the at least one branched, liquid alkane with carbon chain length of C12 to C20 comprises isohexadecane;
iii. the at least one emollient ester, ether, or carbonate with carbon chain length of C12 to C24 comprises one of isopropyl palm itate, and ethylhexyl palm itate; and
iv. the at least one hydrating agent comprises glycerin.

In some embodiments, the composition comprises at least two silicone-based emulsifiers, each present in the composition from about 0.1% to about 20%, by weight of the composition. In some embodiments, the at least two silicone-based emulsifiers are chosen from dimethicone/PEG-10/15 crosspolymer, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and lauryl PEG-9 polydimethylsiloxyethyl dimethicone, and combinations thereof.

In some embodiments, the composition comprises at least one additive. In some embodiments, the at least one additive is present up to about 0.5% by weight of the composition.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

It is to be understood that both the foregoing general description and the following detailed description are exemplary and explanatory only and are not restrictive of the disclosure.

### BRIEF DESCRIPTION OF THE DRAWINGS

Features and advantages of the general inventive concepts will become apparent from the following detailed description made with reference to the accompanying drawings.

FIG. 1 is a data plot in the form of a 2D graph that shows the makeup removal efficacy of inventive compositions according to the disclosure and comparative compositions.

### DETAILED DESCRIPTION

“Cosmetically acceptable” means a carrier that is compatible with any keratinous substrate.

“Keratinous substrate” and “keratinous tissue” each includes but is not limited to skin, hair, and nails.

The composition according to the disclosure includes at least one of each of a silicone-based emulsifier, a branched, liquid alkane with carbon chain length of C12 to C20, an emollient ester, ether, or carbonate with carbon chain length of C12 to C24, a hydrating agent, and water. In some embodiments, the composition may further include one or more additives including preservatives, actives, and fragrances.

The composition according to the disclosure is a water-in-oil (as exemplified herein, a silicone oil) emulsion. A water-in-oil emulsion is sometimes referred to as a reverse emulsion in comparison with the more common type of oil-in-water emulsion skin cleanser formulations. The composition has a cream-gel texture that demonstrates medium to long playtime, providing a pleasant feel when applied and does not immediately absorb into the skin or evaporate. Upon massaging onto the face, the composition demonstrates effective dissolving of eye, face and lip makeup. Using a tissue or a cotton round allows the user to gently wipe away the dissolved makeup products. The composition does not require rinsing after application and leaves the skin with a silky and hydrated feel that is not greasy or sticky.

According to the disclosure, the inventive composition overcomes the shortcomings of the prior art and provide unexpected benefits that include ease with overall makeup removal and easy foundation removal and easy mascara removal (regular and waterproof) with no rubbing, with overall improved efficacy as compared with commercial comparative compositions, leaves skin feeling clean and fresh, soft and smooth, is non-greasy and does not leave a residue, has a pleasant fragrance.

Through expert evaluations of makeup removal efficacy, exemplified embodiments of the composition according to the disclosure demonstrated significantly better performance in removing both mascara, including waterproof mascara) and long-wear foundation as compared with currently marketed MURs. The exemplified embodiments of the inventive composition according to the disclosure not only performed better in makeup removal efficacy, including improved performance in the removal of both mascara and foundation, they also provided an experience that was more pleasing and resulted in comparatively better skin feel after use.

(i) Silicone-Based Emulsifiers

In accordance with the disclosure, embodiments of the composition include at least one silicone-based emulsifier. In some embodiments, the composition comprises more than one silicone-based emulsifier. In some embodiments, the composition includes two or more silicone-based emulsifiers, wherein the two silicone-based emulsifiers may differ from one another in that one emulsifier has a greater chemical molecular weight than the other emulsifier.

In some embodiments, suitable silicone-based emulsifiers may be chosen from silicone emulsifiers that include functional groups chosen from polyglyceryl, polyethylene glycol, or polypropylene glycol. wherein such silicone-based emulsifiers may be chosen from dimethicone/PEG-10/15 crosspolymer, dimethicone/polyglycerin-3 crosspolymer, polyether-modified crosslinked silicone polymers in dimethicone, including, for example, but not limited to PEG-15/lauryl polydimethylsiloxyethyl dimethicone crosspolymer.

In some embodiments, suitable silicone-based emulsifiers may be chosen from alkyldimethicone copolyol type and dimethicone copolyol type silicone emulsifiers. The at least one silicone-based emulsifier may be chosen from dimethicone/PEG-10/15 crosspolymer, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and lauryl PEG-9 polydimethylsiloxyethyl dimethicone, and combinations thereof.

In some particular embodiments according to the disclosure, the composition comprises a silicone-based emulsifier comprising dimethicone/PEG-10/15 crosspolymer, alone or in combination with PEG-10 dimethicone.

In various embodiments, each of the at least one silicone-based emulsifier is present in the composition at a concentration, from about 0.1% to about 20%, and in some embodiments, from about 0.2% to about 15%, and in some embodiments, from about 0.3% to about 10%, and in some embodiments, from about 1% to about 5%, and in some embodiments, from about 2% to about 4%, or any suitable combination, sub-combination, range, or sub-range thereof by weight, based on the weight of the composition. In some embodiments, the composition comprises more than one silicone-based emulsifier, the combination thereof present in the composition at a concentration, from about 0.2% to about 20%, and each of the more than one present in the composition at a concentration from about 0.1% to about 20%, or any suitable combination, sub-combination, range, or sub-range thereof by weight, based on the weight of the composition. One of ordinary skill in the art, however, will appreciate that other ranges are within the scope of the invention.

In some representative examples, a silicone-based emulsifier comprising dimethicone/PEG-10/15 crosspolymer may be present in the composition in a range from about 1% to about 4% or at about 3% together with PEG-10 dimethicone present in the composition in a range from about 0.1% to about 0.5% or at about 0.2%.

Thus, in various embodiments, each of the at least one silicone-based emulsifier is present in a composition according to the disclosure from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 to about 20 percent by weight, including increments and ranges there between.

(ii) Branched, Liquid Alkane

In accordance with the disclosure, embodiments of the composition include at least one branched, liquid alkane. In some embodiments, the composition comprises more than one branched, liquid alkane. In some embodiments, the branched, liquid alkane has a carbon chain length of C12 to C20.

In some embodiments, the branched, liquid alkane has a carbon chain length of C12 to C20. The branched, liquid alkane may be chosen from those with a carbon chain length of from C12 to C20, from C13-C15, from C15 to C19, and C12, C13, C14, C15, C16, C17, C18, C19 and C20. In some particular embodiments, the branched, liquid alkane may be chosen from isoparaffins, for instance isododecane (also known as 2,2,4,4,6-pentamethylheptane), isodecane, C15-19 alkane, and isohexadecane.

In some particular embodiments the at least one branched, liquid alkane comprises isohexadecane.

In various embodiments, the at least one branched, liquid alkane is present in the composition at a concentration, from about 1% to about 15%, and in some embodiments, from about 2% to about 10%, and in some embodiments, from about 2% to about 5% or any suitable combination, sub-combination, range, or sub-range thereof by weight, based on the weight of the composition. In some embodiments, the composition comprises more than one branched, liquid alkane, the combination thereof present in the composition at a concentration, from about 5% to about 10%, and each of the more than one present in the composition at a concentration from about 2% to about 5% or any suitable combination, sub-combination, range, or sub-range thereof by weight, based on the weight of the composition. One of ordinary skill in the art, however, will appreciate that other ranges are within the scope of the invention.

Thus, in various embodiments, each of the at least one branched, liquid alkane is present in a composition according to the disclosure from about 1, 2, 3, 4, 5, 6, 7, 8, 9, 10, 11, 12, 13, 14 to about 15 percent, by weight, including increments and ranges therein and there between.

(iii) Emollient Ester, Ether or Carbonate

In accordance with the disclosure, embodiments of the composition include at least one emollient ester, ether, or carbonate. In some embodiments, the composition comprises more than one emollient ester, ether, or carbonate. The emollient ester, ether, or carbonate has a carbon chain length of C12 to C24. In some embodiments, the at least one emollient ester is an alkylated alcohol ester.

In some embodiments, suitable emollient ester, ether, or carbonates that may be used according to the disclosure may be chosen from isopropyl myristate, isopropyl palm itate, ethylhexyl palm itate, methyl palm itate, 2-ethylhexyl palm itate, hexyl laurate, ethylhexyl laurate, isopropyl laurate, ethylhexyl oleate, ethylhexyl isononanoate, isononyl isononanoate, myristyl myristate, 2-ethylhexyl caprate/caprylate (or octyl caprate/caprylate), isostearyl neopentanoate, cetearyl ethylhexanoate, isopropyl isostearate, diisopropyl sebacate, coco caprylate/caprate, diisopropyl adipate, and combinations thereof. In some particular embodiments, the at least one emollient ester, ether or carbonate is selected from ethylhexyl palmitate, isopropyl palmitate, dicaprylyl ether, dicaprylyl carbonate, isopropyl myristate and combinations thereof.

In some embodiments, the emollient ester, ether, or carbonate is an emollient ester comprising esters of lactic acid and fatty alcohols and comprising 12 or 13 carbon atoms, and combinations thereof.

In some particular embodiments the at least one emollient ester, ether, or carbonate branched is an alkylated alcohol ester. In some embodiments the composition comprises two or more branched, alkylated alcohol esters. In some such embodiments, the at least one branched, alkylated alcohol ester comprises isopropyl myristate and optionally one or more additional branched, alkylated alcohol ester.

In some particular embodiments, the composition comprises one or a combination of ethylhexyl palmitate, isopropyl palmitate, dicaprylyl ether, dicaprylyl carbonate and isopropyl myristate. In some particular embodiments, the composition comprises only one of ethylhexyl palmitate, isopropyl palmitate, dicaprylyl ether, or dicaprylyl carbonate, or a combination of isopropyl palm itate with one of dicaprylyl ether and dicaprylyl carbonate.

In various embodiments, the at least one emollient ester, ether, or carbonate is present from about 0.5% to about 10% by weight of the composition, and in some embodiments, from about 0.8% to about 8%, and in some embodiments, from about 1% to about 7%, and in some embodiments, from about 2% to about 5%, and in some embodiments, about 5% or about 2%, or any suitable combination, sub-combination, range, or sub-range thereof by weight, based on the weight of the composition. In some embodiments, the composition comprises more than one emollient ester, ether, or carbonate, the combination thereof present in the composition at a concentration, from about 1% to about 20%, and each of the more than one present in the composition at a concentration from about 0.5% to about 10%, or any suitable combination, sub-combination, range, or sub-range thereof by weight, based on the weight of the composition. One of ordinary skill in the art, however, will appreciate that other ranges are within the scope of the invention.

Thus, in various embodiments, each one or a combination of the at least one emollient ester, ether, or carbonate is present in a composition according to the disclosure from about 0.5, 0.6. 0.7, 0.8, 0.9, 1, 2, 3, 4, 5, 6, 7, 8, 9,10, 11, 12, 13, 14, 15, 16, 17, 18, 19, to about 20 percent, by weight, including increments and ranges therein and there between.

(iv) Hydrating Agent

In accordance with the disclosure, embodiments of the composition include at least one hydrating agent. In some embodiments, the composition comprises more than one hydrating agent. The hydrating agent may be a polyol.

In some embodiments, suitable hydrating agents that may be used according to the disclosure may be chosen one or more of polyols, including, for example, glycerin, glycerol, glycols, such as caprylyl glycol, butylene glycol, propylene glycol, isoprene glycol, dipropylene glycol, hexylene glycol and polyethylene glycols, monoethylene glycol, diethylene glycol, diethylene glycol, diethylene glycol, hexylene glycol; glycol ethers such as monopropylene, dipropylene and tripropylene glycol alkyl(C1-C4)ethers; squalane; triacetin; sugars, such as glucose, xylitol, maltitol, sorbitol, sucrose pentaerythritol, inositol; pyrrolidone carboxylic acid (PCA); lactic acid; lithium chloride; acetamide MEA; sodium lactate; urea; dicyanamide; hyaluronic acid; aloe vera; honey; seaweed extract; and combinations thereof.

In some embodiments, the hydrating agent comprises glycerin.

In various embodiments, the at least one hydrating agent is present from about 5% to about 30% by weight of the composition, and in some embodiments, from about 5% to about 25%, and in some embodiments, from about 10% to about 20%, and in some embodiments, about 15%, or any suitable combination, sub-combination, range, or sub-range thereof by weight, based on the weight of the composition. In some embodiments, the composition comprises more than one hydrating agent, the combination thereof present in the composition at a concentration, from about 5% to about 30%, and each of the more than one present in the composition at a concentration from about 5% to about 25%, and in some embodiments, about 15% or any suitable combination, sub-combination, range, or sub-range thereof, by weight, based on the weight of the composition. One of ordinary skill in the art, however, will appreciate that other ranges are within the scope of the invention.

Thus, in various embodiments, each of the at least one emollient ester, ether, or carbonate is present in a composition according to the disclosure from about 5, 6, 7, 8, 9, 10, 11, 12,13, 14, 15, 16, 17, 18, 19, 20, 21, 22, 23, 24, 25, 26, 27, 28, 29, to about 30 percent, by weight, including increments and ranges therein and there between.

v Water

In accordance with the various embodiments, water is present in the compositions in a range from about 60% to about 85%, and in some embodiments, from about 65% to about 85%, and in some embodiments, from about 65% to about 75%, and in some embodiments, from about 66% to about 72%, and in some embodiments, about 68% or any suitable combination, sub-combination, range, or sub-range thereof by weight, based on the weight of the composition. One of ordinary skill in the art, however, will appreciate that other ranges are within the scope of the invention. Thus, water is present, by weight, based on the total weight of the composition, from about 60, 61, 62, 63, 64, 65, 66, 67, 68, 69, 70, 71, 72, 73, 74, 75, 76, 77, 78, 79, 80, 81, 82, 83, 84, to about 85 percent, by weight, including increments and ranges therein and there between. One of ordinary skill in the art, however, will appreciate that other ranges are within the scope of the invention.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the composition may be adjusted prior to combining the oil phase with the water phase to avoid the practical difficulty with measuring pH in an internal water phase of the water-in-oil emulsion. Accordingly, the pH of the water phase prior to emulsification can be adjusted with pH adjusters to a pH in a range from about 2 to about 12, and in some embodiments, is from about 3 to about 11, and from about 4 to about 9, and from about 5 to about 8, and in some embodiments about 5.5. The pH of the water phase can be adjusted by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

Optional Additives

The compositions may also comprise at least one additive used in the cosmetics field which does not affect the properties of the compositions according to the invention, such as fragrances, preservatives/anti-microbials (for example, potassium sorbate, and caprylyl glycol); actives (for example, hydroxyacetophenone, vitamins, panthenol, tocopherol); coloring materials; essential oils; citric acid, sodium citrate, sodium chloride; neutralizing, chelating or pH-adjusting agents (for example, triethylamine (TEA), trisodium ethylenediamine disuccinate, and sodium hydroxide), and combinations thereof.

Although the optional additives are given as examples, it will be appreciated that other optional components compatible with cosmetic applications known in the art may be used that are suitable for a cosmetic composition.

In some particular embodiments, the composition may include optional additives selected chose from sodium citrate, sodium chloride, hydroxyacetophenone sodium benzoate, potassium sorbate, citric acid, caprylyl glycol, and trisodium ethylenediamine disuccinate, and combinations thereof.

In accordance with the various embodiments, the amount of one or more actives and additives, alone or in combination, when present in the composition according to the disclosure can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5%and in some embodiments, from about 1% to about 2%, or any suitable combination, sub-combination, range, or sub-range thereof by weight, based on the total weight of the composition. One of ordinary skill in the art, however, will appreciate that other ranges are within the scope of the invention.

Thus, any one or a combination of actives and additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

The examples below according to the invention are given by way of illustration and without a limiting nature. The names are the chemical name or the INCI name. The amounts are given therein as % by weight, unless otherwise mentioned.

### EXAMPLES

### Example 1

### Inventive Compositions

Various representative embodiments of the inventive compositions and articles are exemplified herein.

TABLE 1
Inventive Compositions

| | | Inventive Examples | | | | |
|---|---|---|---|---|---|---|
| Phase | INCI Name | Ex 1. | Ex 2. | Ex 3. | Ex 4. | Ex 5. |
| A | DIMETHICONE (and) | 3 | 3 | 3 | 3 | 3 |
| | DIMETHICONE/PEG-10/15 | | | | | |
| | CROSSPOLYMER (75/25) | | | | | |
| A | PEG-10 DIMETHICONE | 0.2 | 0.2 | | | |
| A | ISOHEXADECANE | 7 | 7 | | | |
| A | ISODODECANE | | | 7 | | |
| A | C15-19 ALKANE | | | | 5 | 5 |
| A | ETHYLHEXYL PALMITATE | 5 | | 5 | | |
| A | ISOPROPYL PALMITATE | | 5 | | 5 | 5 |
| A | DICAPRYLYL ETHER | | | | 2 | |
| A | DICAPRYLYL CARBONATE | | | | | 2 |
| B | WATER (QS) | 68.72 | 68.12 | 68.32 | 68.32 | 68.32 |
| B | GLYCERIN | 15 | 15 | 15 | 15 | 15 |
| B | HYDROXYACETOPHENONE | | 0.5 | 0.5 | 0.5 | 0.5 |
| B | CAPRYLYL GLYCOL | | 0.3 | 0.3 | 0.3 | 0.3 |
| B | TRISODIUM | | 0.1 | 0.1 | 0.1 | 0.1 |
| | ETHYLENEDIAMINE | | | | | |
| | DISUCCINATE | | | | | |
| B | SODIUM BENZOATE | 0.2 | | | | |
| B | POTASSIUM SORBATE | 0.1 | | | | |
| B | Sodium Citrate | 0.2 | 0.2 | 0.2 | 0.2 | 0.2 |
| B | Sodium Chloride | 0.5 | 0.5 | 0.5 | 0.5 | 0.5 |
| B | Citric Acid | 0.08 | 0.08 | 0.08 | 0.08 | 0.08 |
| | Total (%): | 100 | 100 | 100 | 100 | 100 |

### Example 2

### Comparative Compositions

TABLE 2
Comparative Composition 1

| Phase | INCI Name | Wt % |
|---|---|---|
| A | DIMETHICONE (and) DIMETHICONE/PEG-10/15 | 5 |
| | CROSSPOLYMER (75/25) | |
| A | Dimethicone | 7 |
| B | Water (QS) | 66.92 |
| B | Glycerin | 15 |
| B | Propanediol | 5 |
| B | SODIUM BENZOATE | 0.2 |
| B | POTASSIUM SORBATE | 0.1 |
| B | Sodium Citrate | 0.2 |
| B | Sodium Chloride | 0.5 |
| B | Citric Acid | 0.08 |
| | Total (%): | 100 |

TABLE 3
Comparative Composition 2 - Lancôme Galatéis
Douceur (Gentle Cleanser for Face and Eyes), a commercially
available emulsion (cream)-based makeup removing product
Ingredients

AQUA/WATER
PARAFFINUM LIQUIDUM/MINERAL OIL
ISOPROPYL MYRISTATE
PROPANEDIOL
CARBOMER
PEG-32
PEG-60 HYDROGENATED CASTOR OIL
SALICYLIC ACID
DISODIUM EDTA
PARFUM/FRAGRANCE
SODIUM HYDROXIDE
DISODIUM COCOAMPHODIACETATE
LINALOOL
HYDROXYCITRONELLAL
BENZYL ALCOHOL
CINNAMYL ALCOHOL
GERANIOL
ANANAS SATIVUS FRUIT EXTRACT/PINEAPPLE FRUIT EXTRACT
HEXYL CINNAMAL
EUGENOL
PAPAIN
CITRONELLOL
CI 77007/ULTRAMARINES
SILICA

TABLE 4
Comparative Composition 3 - NEUTROGENA
Makeup Remover Cleansing Towelettes
Ingredients

Water
Isononyl Isononanoate
Isostearyl Palmitate
Cetyl Ethylhexanoate
Cyclopentasiloxane
Pentaerythrityl Tetraethylhexanoate
Hexylene Glycol
PEG-6 Caprylic/Capric Glycerides
Phenoxyethanol
Sucrose Cocoate
Carbomer
PEG-4 Laurate
Fragrance
Sodium Hydroxide
Benzoic Acid
Dehydroacetic Acid
Iodopropynyl Butylcarbamate
Ethylhexylglycerin

TABLE 5
Comparative Composition 4 - GARNIER SkinActive Micellar
Cleansing Water All-in-1 Cleanser & Makeup Remover (Mono-phase)
Ingredients

AQUA/WATER
HEXYLENE GLYCOL
GLYCERIN
POLOXAMER 184
DISODIUM COCOAMPHODIACETATE
DISODIUM EDTA
POLYAMINOPROPYL BIGUANIDE

TABLE 6
Comparative Composition 5 - GARNIER SkinActive Micellar Cleansing
Water All-in-1 Cleanser & Waterproof Makeup Remover (Bi-Phase)
Ingredients

WATER/AQUA
CYCLO PENTASILOXANE
ISOHEXADECANE
POTASSIUM PHOSPHATE
HEXYLENE GLYCOL
POLYAMINOPROPYL BIGUANIDE
SODIUM CHLORIDE
DIPOTASSIUM PHOSPHATE
DECYL GLUCOSIDE
DISODIUM EDTA

### Example 3

### Expert Evaluation

Demonstration of the effectiveness of inventive compositions vs comparative compositions in stubborn makeup and mascara removal.

A study was conducted to evaluate the effectiveness of the inventive compositions in comparison with commercially available MURs (comparative compositions), having generally similar components. In the study, the tested compositions were used by trained/expert aesthetician to remove heavy face makeup (Maybelline (™) Super Stay 24 hour Full Coverage Foundation), and mascara (Maybelline (™) Colossal Big Shot Mascara and Express Waterproof Mascara).

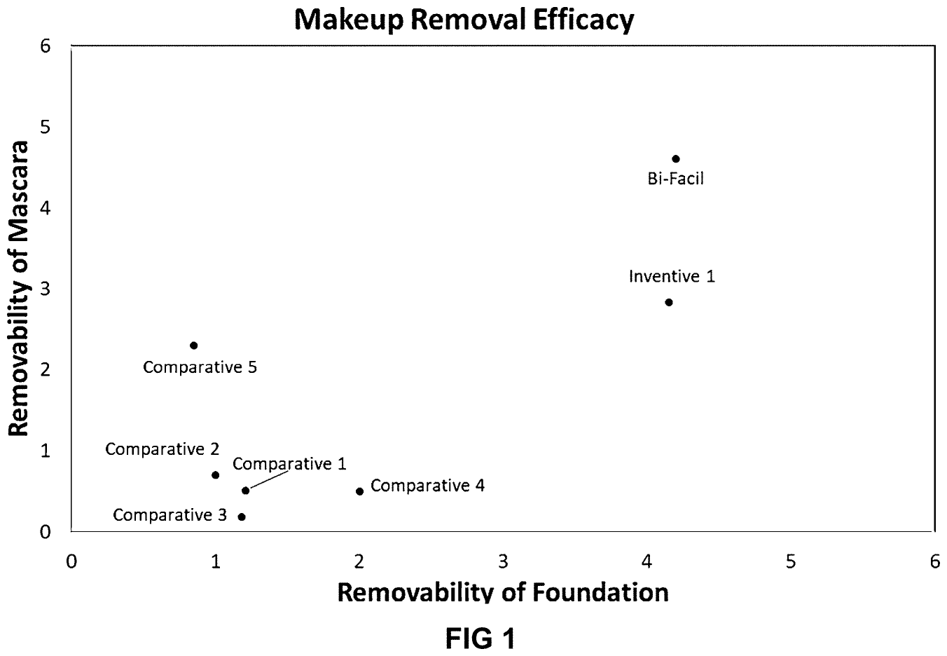

The makeup removal efficacy was determined by following a protocol under which the aesthetician applied stubborn makeup products on six (6) Caucasian women. After 30 min, the aesthetician removed the makeup with test products (inventive and comparative), using 3 cotton rounds each soaked with 2 ml of the test composition for mascara removal, and using 2 cotton rounds each soaked with 2 ml of the test composition for foundation removal. The degree of removal was assessed by the amount of residual makeup that was removed by using Lancome (™) Bi-Facil (™) Makeup Remover (a two-phase make-up removal product), 2 mL on a cotton pad for up to 4 cotton pads. The higher the makeup removal efficacy of the test formula would require fewer cotton pads with Bi-Facil (™) to remove, as there would be less residual makeup. The makeup removal efficacy data was plotted using a 2D graph, shown as FIG. 1; the data as shown in FIG. 1 are represented in Table 7.

TABLE 7
Make-Up Removal Efficacy (scale of 0 to 6:6 being
complete removal, and 0 being no removal)

| Test Composition | Foundation | Mascara |
|---|---|---|
| Inventive Ex. 1 | 4.2 | 2.8 |
| Comparative Ex.1 | 1.2 | 0.5 |
| Lancôme Galatéis Douceur (Comparative Ex. 2) | 1.0 | 0.7 |
| Neutrogena Cleansing Wipes (Comparative Ex. 3) | 1.2 | 0.2 |
| Gamier Micellar Water, Mono-phase (Comparative Ex. 4) | 2.0 | 0.5 |
| Gamier Micellar Water, Bi-phase (Comparative Ex. 5) | 0.9 | 2.3 |

As visually represented in FIG. 1, the inventive compositions each fall within the “most effective MUR” category for removing stubborn makeup products. This is the same category of Lancôme Bi-Facil, compared to which the inventive compositions lack a greasy after-feel typically found with use of Bi-Facil.

While the disclosure has been described with reference to described embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The terms “weight percent” and “wt %” may be used interchangeably and mean percent by weight, based on the total weight of a composition, article or material, except as may be specified with respect to, for example, a phase, or a system that is a component of a composition, article or material. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A makeup removal composition, comprising:
i. at least one silicone-based emulsifier;
ii. at least one branched, liquid alkane;
iii. at least one emollient ester, ether, or carbonate;
iv. at least one hydrating agent; and
v. water.

2. The makeup removal composition according to claim 1, wherein: the at least one branched, liquid alkane has a carbon chain length of C12 to C20; and wherein the at least one emollient ester, ether, or carbonate has a carbon chain length of C12 to C24.

3. The makeup removal composition according to claim 1, the composition comprising more than one of the silicone-based emulsifier, the emollient ester, ether or carbonate, or a combination thereof.

4. The makeup removal composition according to claim 1, wherein the at least one silicone-based emulsifier comprises dimethicone/PEG-10/15 crosspolymer, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and lauryl PEG-9 polydimethylsiloxyethyl dimethicone, or a combination thereof.

5. The makeup removal composition according to claim 1, wherein the at least one silicone-based emulsifier comprises dimethicone/PEG-10/15 crosspolymer or dimethicone/PEG-10/15 crosspolymer and PEG-10 dimethicone.

6. The makeup removal composition according to claim 1, wherein the at least one silicone-based emulsifier is present from about 0.5% to about 20%, by weight of the composition.

7. The makeup removal composition according to claim 1, wherein at least one branched, liquid alkane comprises isohexadecane, isododecane, C15-19 alkane, or a combination thereof.

8. The makeup removal composition according to claim 1, wherein the at least one branched, liquid alkane is present from about 1% to about 15% by weight of the composition.

9. The makeup removal composition according to claim 1, wherein the at least one emollient ester, ether, or carbonate comprises a dialkylated ether or a dialkylated carbonate.

10. The makeup removal composition according to claim 1, wherein the at least one emollient ester, ether, or carbonate comprises isopropyl myristate, isopropyl palmitate, ethylhexyl palmitate, dicaprylyl ether, dicaprylyl carbonate, or a combination thereof.

11. The makeup removal composition according to claim 1, wherein the at least one emollient ester, ether, or carbonate is present from about 0.5% to about 10% by weight of the composition.

12. The makeup removal composition according to claim 1, wherein the at least one hydrating agent is present from about 10% to about 20% by weight of the composition.

13. A makeup removal composition, comprising:
i. at least one silicone-based emulsifier present from about 0.5% to about 20%, by weight of the composition;
ii. at least one branched, liquid alkane present from about 1% to about 15% by weight of the composition;
iii. at least one emollient ester, ether, or carbonate present from about 0.5% to about 10% by weight of the composition;
iv. at least one hydrating agent present from about 10% to about 20% by weight of the composition; and
v. water present from about 60% to about 85% by weight of the composition.

14. The makeup removal composition according to claim 13, wherein:
i. the at least one silicone-based emulsifier comprises dimethicone/PEG-10/15 crosspolymer, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and lauryl PEG-9 polydimethylsiloxyethyl dimethicone, or a combination thereof;
ii. the at least one branched, liquid alkane has a carbon chain length of C12 to C20;
iii. the at least one emollient ester, ether, or carbonate has a carbon chain length of C12 to C24 and comprises a dialkylated ether or a dialkylated carbonate; and
iv. the at least one hydrating agent comprises a polyol.

15. The makeup removal composition according to claim 13, wherein:
i. the at least one silicone-based emulsifier comprises (a) dimethicone/PEG-10/15 crosspolymer, dimethicone/polyglycerin-3 crosspolymer, cetyl PEG/PPG-10/1 dimethicone, PEG-18/PPG-18 dimethicone, or a combination thereof; or (b) (1) dimethicone/PEG-10/15 crosspolymer, dimethicone/polyglycerin-3 crosspolymer, cetyl PEG/PPG-10/1 dimethicone, PEG-18/PPG-18 dimethicone, or a combination thereof and (2) PEG-10 dimethicone, PEG/PPG-18/18 methicone, lauryl PEG/PPG-18/18 methicone, PEG-9 polydimethylsiloxyethyl dimethicone, lauryl PEG-9 polydimethylsiloxyethyl dimethicone, or a combination thereof;
ii. the at least one branched, liquid alkane has a carbon chain length of C12 to C20 and comprises isohexadecane, isododecane, C15-19 alkane, or a combination thereof;
iii. the at least one emollient ester, ether, or carbonate has a carbon chain length of C12 to C24 and comprises isopropyl myristate, isopropyl palmitate, ethylhexyl palmitate, dicaprylyl ether, dicaprylyl carbonate, or a combination thereof;
iv. the at least one hydrating agent comprises glycerin.

16. The makeup removal composition according to claim 1, wherein
i. the at least one silicone-based emulsifier comprises dimethicone/PEG-10/15 crosspolymer;
ii. the at least one branched, liquid alkane comprises isododecane or C15-19 alkane;
iii. the at least one emollient ester, ether, or carbonate with carbon chain length of C12 to C24 comprises isopropyl palm itate, ethylhexyl palmitate, dicaprylyl ether, dicaprylyl carbonate, a combination of isopropyl palm itate and dicaprylyl ether, or a combination of isopropyl palm itate and dicaprylyl carbonate; and
iv. the at least one hydrating agent comprises glycerin.

17. The makeup removal composition according to claim 1, wherein
i. the at least one silicone-based emulsifier comprises dimethicone/PEG-10/15 crosspolymer and PEG-10 dimethicone;
ii. the at least one branched, liquid alkane with carbon chain length of C12 to C20 comprises isohexadecane;
iii. the at least one emollient ester, ether, or carbonate with carbon chain length of C12 to C24 comprises isopropyl palm itate, ethylhexyl palm itate, or a combination thereof; and
iv. the at least one hydrating agent comprises glycerin.

18. The makeup removal composition according to claim 1, wherein
i. the at least one silicone-based emulsifier is present from about 0.5% to about 20%, by weight of the composition;
ii. the at least one branched, liquid alkane present is from about 1% to about 15% by weight of the composition;
iii. the at least one emollient ester, ether, or carbonate is present from about 0.5% to about 10% by weight of the composition;
iv. the at least one hydrating agent is present from about 10% to about 20% by weight of the composition; and
v. water is present from about 60% to about 85% by weight of the composition.

19. A method for removing makeup, comprising:
a. selecting a composition comprising:
i. at least one silicone-based emulsifier;
ii. at least one branched, liquid alkane;
iii. at least one emollient ester, ether, or carbonate;
iv. at least one hydrating agent; and
v. water,
wherein the at least one silicone-based emulsifier is present from about 0.5% to about 20%, by weight of the composition, and the at least one branched, liquid alkane is present from about 1% to about 15% by weight of the composition, and the at least one emollient ester, ether, or carbonate is present from about 0.5% to about 10% by weight of the composition, and the at least one hydrating agent is present from about 10% to about 20% by weight of the composition, and water present from about 60% to about 85% by weight of the composition; and
b. applying the composition to a removal article and contacting the composition to make up on the skin in a circular motion followed by swiping the contacted skin to remove the make-up.

20. The method according to claim 19, wherein the method does not require cleansing or water rinsing.
